(19) United States
(12) Patent Application Publication
(10) Pub. No.: US 2021/0308408 A1
(43) Pub. Date: Oct. 7, 2021

(54) MASK WITH FILTER

(71) Applicant: Azimuth Sleep Solutions, Corp., Germantown, TN (US)

(72) Inventor: Kristen Hedger Archbold, Germantown, TN (US)

(21) Appl. No.: 17/222,339

(22) Filed: Apr. 5, 2021

Related U.S. Application Data
(60) Provisional application No. 63/004,805, filed on Apr. 3, 2020.

(51) Int. Cl. A61M 16/10; A61M 16/20; A61M 16/06

(52) U.S. Cl. CPC A61M 16/1065; A61M 16/208; A61M 15/0018; A61M 16/1055; A61M 16/0688

(57) ABSTRACT

A filtered face mask apparatus for CPAP applications can include a body, an inlet port, a mouth portion having a mouth exhaust port, a nose portion having a nose exhaust port, a plurality of adjustable straps, a seal, a mouth exhaust filter disposed over the mouth exhaust port, a nose exhaust filter disposed over the nose exhaust port, and an intake filter disposed over the inlet port. The inlet port may be configured for attachment to a gas supply tube that is attached to a CPAP device on the other end. The mouth exhaust filter, nose exhaust filter, and intake filter may each comprise a N95 or N99 material configured to block the passage of pathogens. A disposable filter patch and a method of operating a positive airway pressure device are also disclosed herein.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### CROSS-REFERENCES TO RELATED APPLICATIONS

This application is a non-provisional of U.S. Patent Application No. 63/004,805 filed Apr. 3, 2020 entitled MASK WITH FILTER, which is hereby incorporated by reference in its entireties.

### STATEMENT REGARDING FEDERALLY SPONSORED RESEARCH OR DEVELOPMENT

Not Applicable.

### REFERENCE TO SEQUENCE LISTING OR COMPUTER PROGRAM LISTING APPENDIX

Not Applicable.

### BACKGROUND

The present disclosure relates generally to masks for healthcare-related applications and more particularly to face masks with inlet and outlet filters to prevent the spread of pathogens.

Conventional face masks such as continuous positive airway pressure (CPAP) masks are commonly used for treatment of sleep disorders such as sleep apnea. Face masks generally form a seal against a user's face and include an inlet port and at least one exhaust port. The inlet port may be coupled to an auxiliary device such as a machine to regulate air pressure provided to a user's face through the mask. Such masks commonly cover a user's nose and mouth during use. A user may breathe normally while wearing the mask, and exhaled air vents out of the mask through one or more exhaust ports.

The exhaled air exiting the mask via an exhaust port may be contaminated with pathogens such as viruses or bacteria if the user is sick. The exhaled air may then contaminate the user's surroundings or other people in the nearby vicinity. This can lead to pathogen spread in the community.

During an epidemic or pandemic, viruses may spread through the air on droplets via contact with nose, mouth, eyes when a patient coughs or sneezes. In such events, conventional personal protective equipment (PPE) such as N95 and N99 masks are in high demand, often leading to shortages in hospitals and for use by the public. Such conventional PPE masks are useful to both prevent healthy persons from becoming sick, and also to prevent sick people from spreading droplets by covering their mouth and nose. When conventional PPE masks are unavailable, pathogens may spread more easily. In such situations, it would be advantageous to repurpose other types of face masks to be used as PPE masks. However, other types of face masks are usually not effective to block virus particles of very small diameter from spreading through the air. Therefore, it is difficult to repurpose face masks of different types, including CPAP face masks, for use as medical PPE masks to block the spread of viruses.

What is needed then are devices and methods for blocking the spread of pathogens using face masks.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

One aspect of the disclosure is a face mask with a filter over the exhaust port to prevent contaminants from a user from passing out of the mask into the surrounding environment. Such contaminants can include pathogens such as a virus or a bacteria, or droplets of a user's body fluid from a cough, sneeze or exhaled breath.

Another aspect of the disclosure is a conventional CPAP mask reconfigured for use as healthcare PPE to prevent contamination of others by a sick patient. The CPAP mask can be repurposed by placing one or more filters over the exhaust port or ports on the mask to prevent passage of pathogens from the user to the surrounding environment.

Another aspect of the disclosure is a face mask modified with an inlet filter to prevent a wearer from being exposed to environmental pathogens such as viruses or bacteria that might otherwise enter the mask via airflow into the mask from the inlet port.

Yet another aspect of the disclosure is a disposable filter patch having an adhesive seal disposed thereon and configured to be universally compatible and applicable to the exhaust ports and inlet ports of any CPAP face mask. When disposed over a CPAP face mask exhaust port, the disposable filter patch would prevent contaminants from a user from passing out of the mask into the surrounding environment. When disposed over a CPAP face mask inlet port, the disposable filter patch prevents a wearer from being exposed to environmental pathogens such as viruses or bacteria that might otherwise enter the mask via airflow into the mask from the inlet port.

Additionally, the examples summarized above can each be incorporated into a method of operating a continuous positive airway pressure device.

Numerous other objects, advantages and features of the present disclosure will be readily apparent to those of skill in the art upon a review of the following drawings and description of a preferred embodiment.

### BRIEF DESCRIPTION OF THE DRAWINGS

FIG. 1 is a perspective view of an embodiment of a mask.

FIG. 2 is a perspective view of another embodiment of a mask.

FIG. 3 is a perspective view of another embodiment of a mask.

FIG. 4 is a perspective view of the mask embodiment of FIG. 1 with a filter positioned on the mouth exhaust port.

FIG. 5 is a perspective view of the mask embodiment of FIG. 2 with a filter positioned on the mouth exhaust port.

FIG. 6 is a side perspective view of the mask embodiment of FIG. 3 secured to a user's face and having a filter positioned on the mouth exhaust port.

FIG. 7 is a perspective view of the mask embodiment of FIG. 1 with a filter positioned on the mouth exhaust port and a filter positioned on the nose exhaust port.

FIG. 8 is a perspective view of the mask embodiment of FIG. 2 with a filter positioned on the mouth exhaust port and a filter positioned on the nose exhaust port.

FIG. 9 is a view of an embodiment of a first and second filter.

FIG. 10 is a view of an embodiment of a first and second filter with adhesive seals.

FIG. 11 is a view of an embodiment of a first and second filter.

FIG. 12 is a view of an embodiment of a first and second filter having equal dimensions.

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

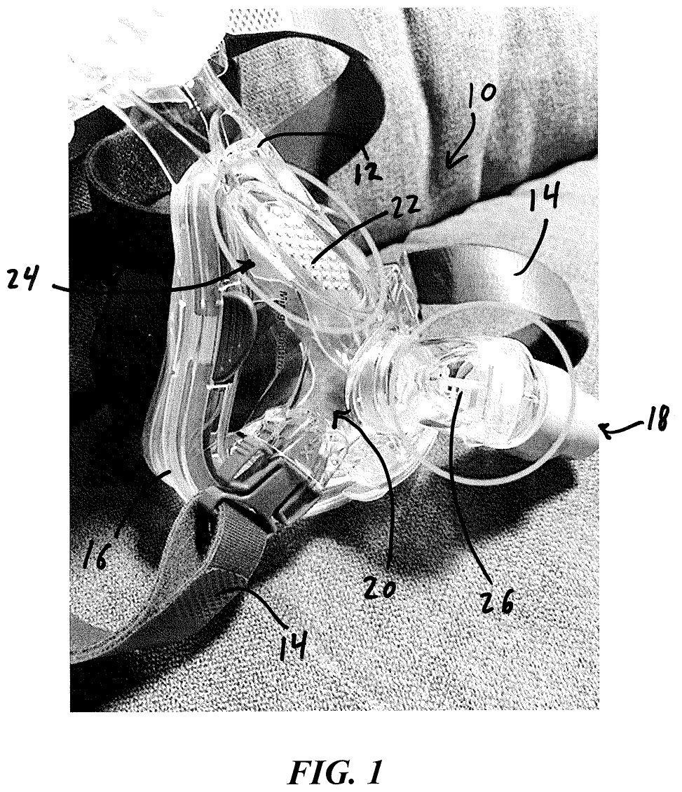

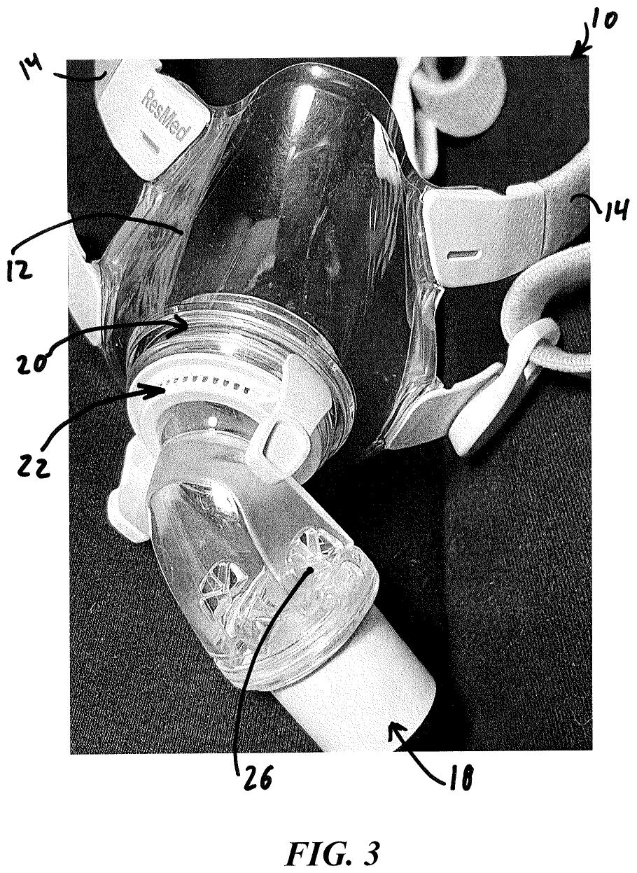

Three distinct embodiments of a conventional CPAP mask are shown in FIGS. 1-3. The face mask 10 includes a body 12 shaped to fit on the front of a user's face. The body 12 includes a mouth region 20 and a nose region 24. The face mask 10 fits tightly against a user's face using strap 14 which extends behind the user's head. In some embodiments, strap 14 can be a plurality of adjustable straps. The face mask 10 also includes a seal 16 which prevents air from escaping between the body 12 and a user's face.

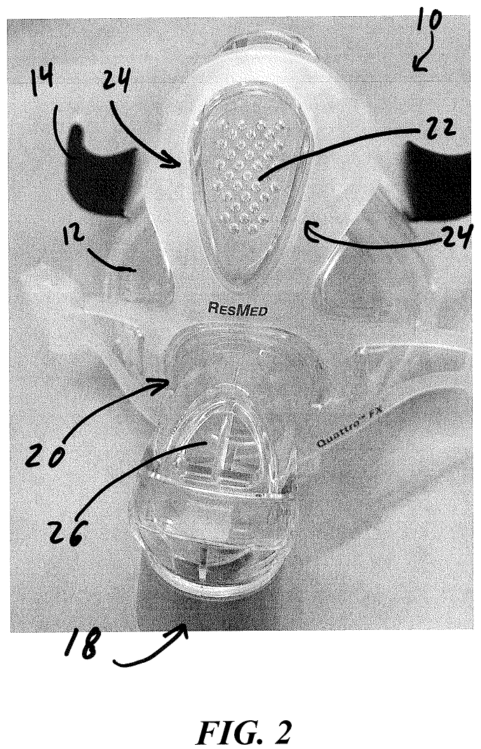

Face mask 10 includes a first exhaust port, or mouth exhaust port 26 positioned adjacent the mouth region 20. In some embodiments, such as those shown in FIGS. 1 and 2, face mask 10 can also include a second exhaust port, or nose exhaust port 22 positioned adjacent the nose region 24. Each exhaust port 22, 26 includes a port through which a user's exhaled breath may escape from the face mask 10. Each exhaust port includes a check valve or directional valve in some embodiments. Each exhaust port 22, 26 is configured to allow exhaled air to escape from the face mask 10 when a user exhales. In embodiments including a check valve, each exhaust port 22, 26 can be configured to allow outside air to enter the face mask 10 when a user inhales.

Face mask 10 also includes an inlet port 18 positioned on body 12 configured for attachment to a tube that supplies a gas such as air or oxygenated air to a user wearing the mask. The tube connected to inlet port 18 extends to a remote device such as a CPAP or BiPAP machine.

Because the face mask 10 covers both the user's mouth and nose, all exhaled air from the user evacuates the mask through either the nose exhaust port 22 or the mouth exhaust port 26, or simultaneously through both. Thus, a user who is positive for a respiratory virus such as a coronavirus, SARS-CoV-2, SARS, MERS, influenza or has any related virus-borne respiratory disease such as COVID-19, would potentially exhale droplets contaminated with the virus through the nose exhaust port 22 or the mouth exhaust port 26. This could contaminate the user's environment or other individuals in the nearby vicinity.

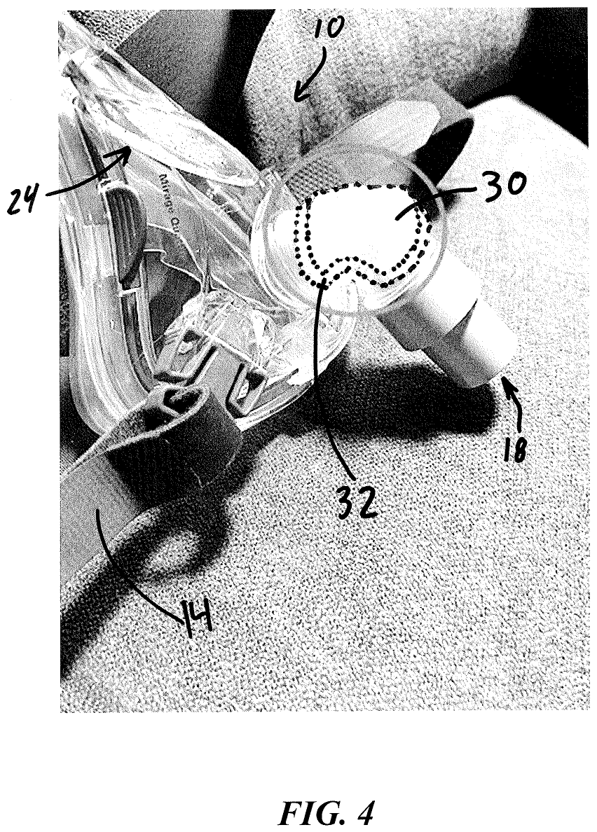

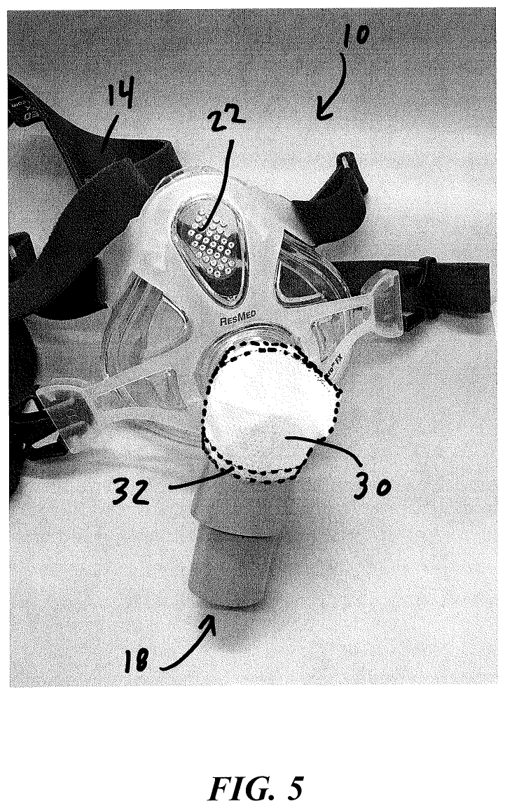

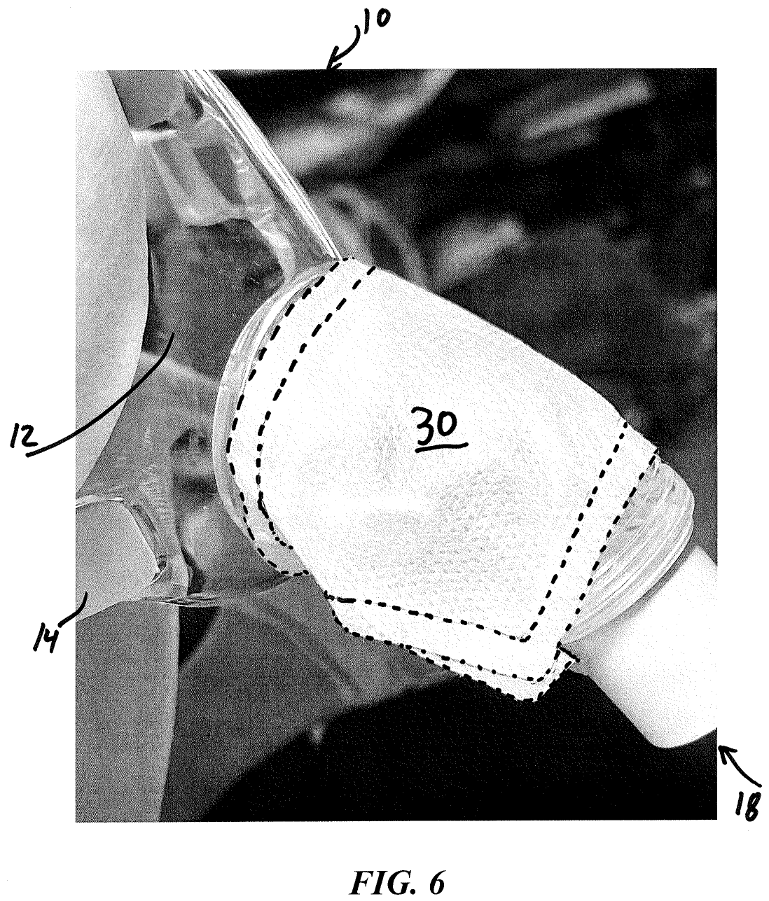

Referring to FIGS. 4-6, in some embodiments, to prevent contaminated exhaled air from exiting the mouth exhaust port 26, a mouth exhaust filter 30 may be positioned over the mouth exhaust port 26. Mouth exhaust filter 30 includes a material suitable for blocking the passage of pathogens, including bacteria and/or viruses. In some embodiments, mouth exhaust filter 30 includes an N95 or N99 material. In other embodiments, mouth exhaust filter 30 includes any particulate air filter capable of removing pathogens and other contaminants from user-expelled breath. The mouth exhaust filter 30 is secured to body 12 using any suitable means, such as but not limited to an adhesive, a wrap, a sleeve, a self-cinching material, a cling adherence, mechanical fasteners, sealing material or any other suitable attachment to secure the mouth exhaust filter 30 to the body 12 covering the mouth exhaust port 26. When mouth exhaust filter 30 is positioned over mouth exhaust port 26 and secured to the body 12, any exhaled air exiting the body via the mouth exhaust port 26 must pass through mouth exhaust filter 30, thereby eliminating the passage of pathogens into the environment.

In some embodiments, mouth exhaust filter 30 includes an adhesive seal 32 disposed around the perimeter of the filter material, allowing mouth exhaust filter 30 to be almost universally applicable to any face mask 10 having a mouth exhaust port 26. As such, the adhesive seal 32 can be pressed against the body 12 around the perimeter of the mouth exhaust port 26, thereby securing the mouth exhaust filter 30 to the mouth exhaust port 26.

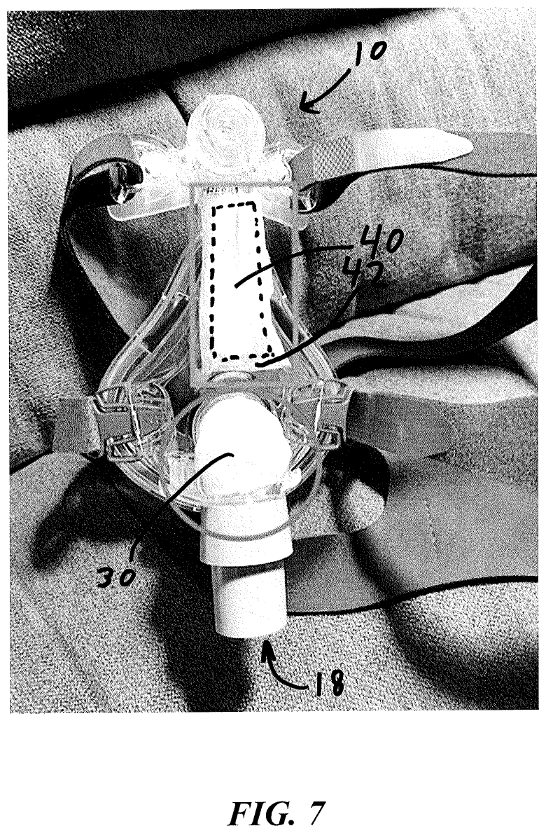

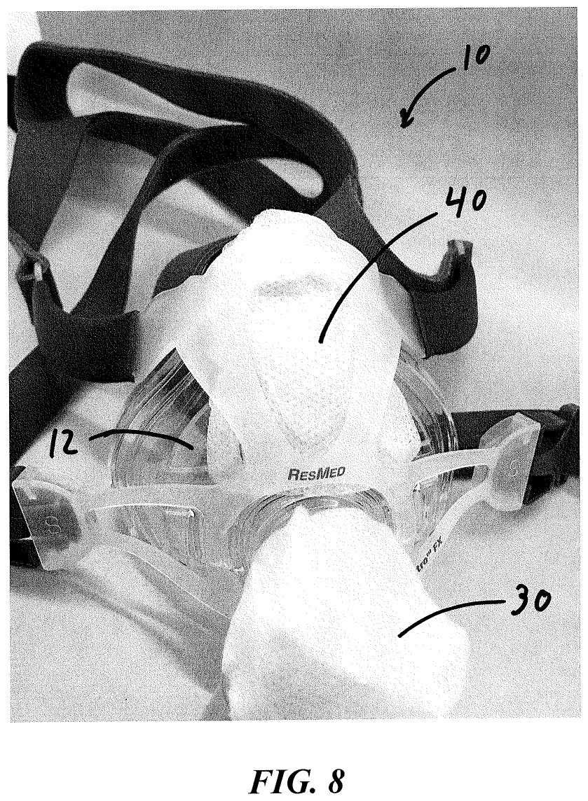

In embodiments having both a mouth exhaust port 26 and a nose exhaust port 22, such as those in FIGS. 7 and 8, in order to prevent contaminated exhaled air from exiting the nose exhaust port 22, a nose exhaust filter 40 may be positioned over the nose exhaust port 22. Nose exhaust filter 40 includes a material suitable for blocking the passage of pathogens, including bacteria and/or viruses. In some embodiments, nose exhaust filter 40 includes an N95 or N99 material. In other embodiments, nose exhaust filter 40 includes any particulate air filter capable of removing pathogens and other contaminants from user-expelled breath. The nose exhaust filter 40 is secured to body 12 using any suitable means, such as but not limited to an adhesive, a wrap, a sleeve, a self-cinching material, a cling adherence, mechanical fasteners, sealing material or any other suitable attachment to secure the nose exhaust filter 40 to the body 12 covering the nose exhaust port 22. When nose exhaust filter 40 is positioned over nose exhaust port 22 and secured to the body 12, any exhaled air exiting the body 12 via the nose exhaust port 22 must pass through nose exhaust filter 40, thereby eliminating the passage of pathogens into the environment.

As shown in FIGS. 7 and 8, in some embodiments, a mouth exhaust filter 30 and a nose exhaust filter 40 are both disposed on body 12 such that both mouth exhaust port 26 and nose exhaust port 22 are covered with a respective filter 30, 40. In such embodiments, all exhaled breath from the user will pass through one of the applied filters 30, 40, thereby preventing the spread of pathogens via exhaled breath, coughing or sneezing of the user.

In some embodiments, nose exhaust filter 40 includes an adhesive seal 42 disposed around the perimeter of the filter material, allowing nose exhaust filter 40 to be almost universally applicable to any face mask 10 having a nose exhaust port 22. As such, the adhesive seal 42 can be pressed against the body 12 around the perimeter of the nose exhaust port 22, thereby securing the nose exhaust filter 40 to the nose exhaust port 22.

In further embodiments, an intake filter can be positioned over the inlet port 18. The intake filter includes a material suitable for blocking the passage of pathogens, including bacteria and/or viruses. In some embodiments, the intake filter includes an N95 or N99 material. In other embodiments, the intake filter includes any particulate air filter capable of removing pathogens and other contaminants from user-expelled breath. The intake filter is secured to body 12, or the hose coupled to the body, using any suitable means, such as but not limited to an adhesive, a wrap, a sleeve, a self-cinching material, a cling adherence, mechanical fasteners, sealing material or any other suitable attachment to secure the intake filter to the body 12 covering the inlet port 18. When inlet filter is positioned over inlet port 18 and secured to the body 12, any incoming air entering the body 12 via the inlet port 18 must pass through intake filter, thereby eliminating the passage of viruses into body 12.

By using an intake filter on inlet port 18, a healthy user may be protected from exposure to environmental pathogens such as viruses or bacteria that might otherwise enter the face mask 10 via inlet port 18.

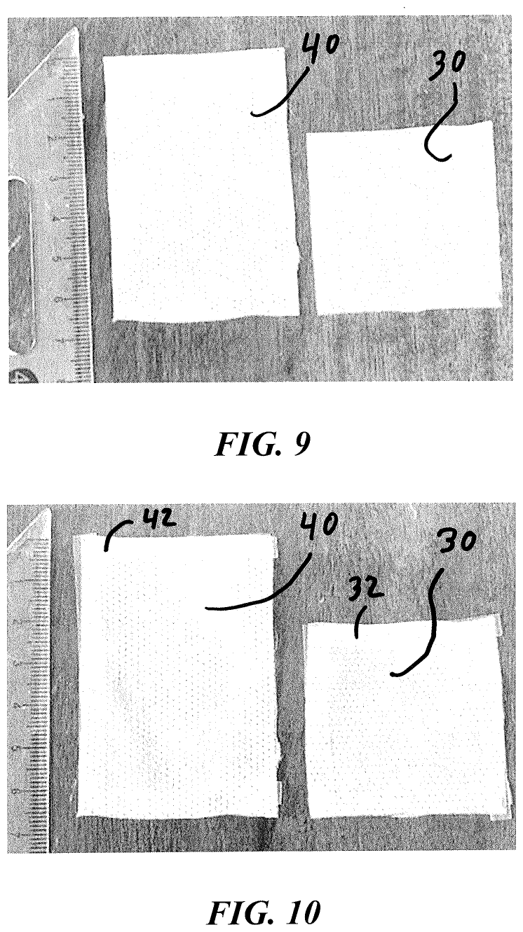

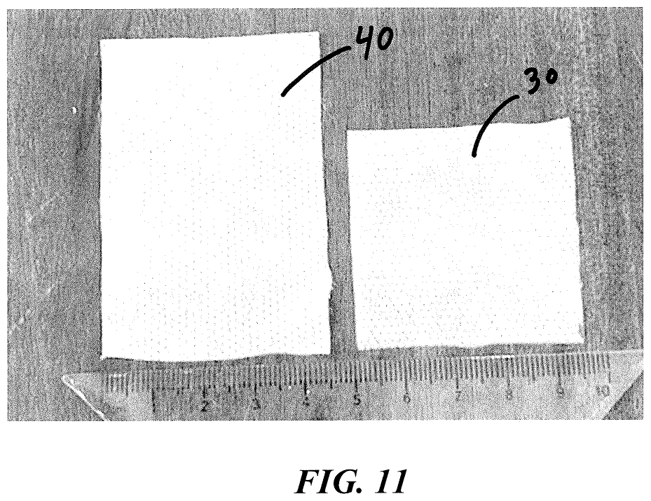

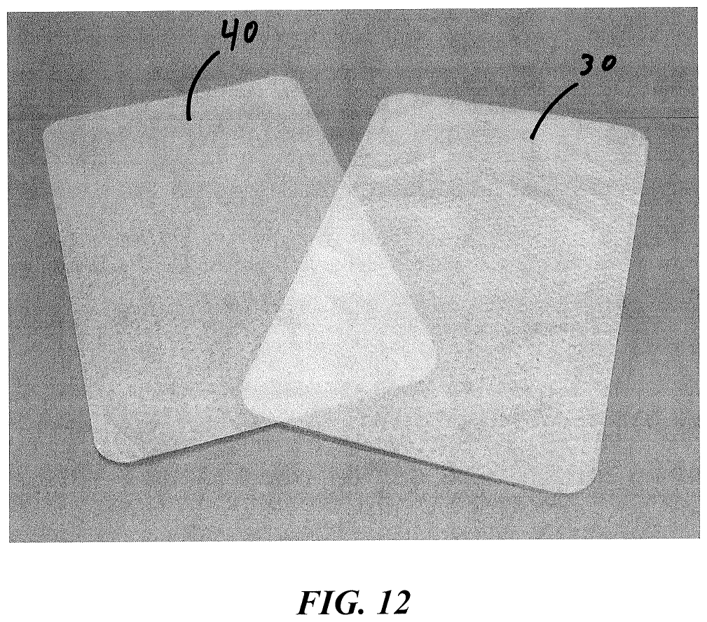

Referring to FIGS. 9-12, additional embodiments of mouth exhaust filter 30 and nose exhaust filter 40 are illustrated. In some embodiments, each filter is between about 1.0 cm and about 10.0 cm in height, and between about 1.0 cm and about 10.0 cm in width. Mouth exhaust filter 30 and nose exhaust filter 40 may each have different dimensions in some embodiments, such as those shown in FIGS. 9-11. In other embodiments, mouth exhaust filter 30 and nose exhaust filter 40 may each have identical dimensions, as shown in FIG. 12. In some embodiments, like that of FIG. 10, mouth exhaust filter 30 includes a first seal 32 disposed on the filter material. First seal 32 can be disposed continuously around the outer edge or perimeter of mouth exhaust filter 30. Similarly, nose exhaust filter 40 includes a second seal 42 disposed on the filter material. Second seal 42 may be disposed continuously around the outer edge or perimeter of nose exhaust filter 40. First seal 32 and second seal 42 may comprise an adhesive seal, such that each filter may be secured to a conventional face mask without any further modification to the face mask, so as to repurpose the face mask for use as personal protective equipment or repurposed to cover the mouth and nose of a contaminated patient.

In further embodiments, mouth exhaust filter 30 may further comprise a first adhesive protector layer that covers first seal 32, and nose exhaust filter 40 may further comprise a second adhesive protector layer that covers second seal 42. In an embodiment, the adhesive protector layer may comprise a think removable film that covers the entire adhesive seal. The thin removable film may be scored along its length or width, so as to enable the film to be more easily torn and peeled away along the scored portion. In this way, the adhesive seal 32 and/or 42 can remain covered and protected until exhaust filter 30 and/or 40 is needed. When needed, users can quickly and easily peel away the adhesive protector layer along the scored portion of the protector layer to reveal the unused adhesive seal underneath. Users may then dispose one or both of mouth and nose exhaust filters 30,40 over exhaust ports 26,22 by pressing the adhesive seal 32,42 against the mask body 12 around the respective perimeter of the mouth or nose exhaust port 26,22.

In some other embodiments, the materials, dimensions, scale, quantities, etc. of the filter can be further modified to accommodate other types of breathing devices to block the passage of pathogens.

Lastly, a method of operating a positive airway pressure device is disclosed. The method may comprise providing a face mask, disposing a mouth exhaust filter over a mouth exhaust port of the face mask, attaching a first end of a gas supply tube to an inlet port of the face mask and the second end of the gas supply tube to a continuous positive airway pressure device, using a set of adjustable straps connected to the face mask to secure the face mask to a user's face such that a face mask seal prevents the escape of air between the user's face and the face mask, and activating the continuous positive airway pressure device such that gas actively flows into the facemask via the inlet port. The method may further include disposing a nose exhaust filter over a nose exhaust port of the face mask and disposing an intake filter over the inlet port of the face mask. The mouth exhaust port and nose exhaust port of the disclosed method may comprise a first check valve and a second check valve, respectively. Additionally, the mouth exhaust filter, the nose exhaust filter, and the intake filter disclosed in the method may comprise a N95 or N99 material.

Thus, although there have been described particular embodiments of the present invention of a new and useful MASK FILTER, it is not intended that such references be construed as limitations upon the scope of this invention.

What is claimed is:

1. A face mask apparatus, comprising:
a body shaped to fit on a user's face;
an inlet port configured for attachment to a gas supply tube;
a mouth portion including a mouth exhaust port; and
a mouth exhaust filter disposed over the mouth exhaust port.

2. The apparatus of claim 1, wherein the mouth exhaust port includes a first check valve.

3. The apparatus of claim 2, further comprising:
a nose portion including a nose exhaust port; and
a nose exhaust filter disposed over the nose exhaust port.

4. The apparatus of claim 3, wherein the mouth exhaust filter and the nose exhaust filter each comprise a particulate air filter capable of removing pathogens and other contaminants from a user's expelled breath.

5. The apparatus of claim 4, wherein the nose exhaust port includes a second check valve.

6. The apparatus of claim 5, further comprising:
a plurality of adjustable straps configured to secure the body to the user's face; and
a seal configured to prevent the escape of air between the body and the user's face.

7. The apparatus of claim 6, further comprising an intake filter disposed over the inlet port.

8. The apparatus of claim 7, wherein the intake filter comprises a particulate air filter capable of removing pathogens and other contaminants from the user's expelled breath.

9. The apparatus of claim 8, wherein each of the mouth exhaust filter, the nose exhaust filter, and the intake filter are removable.

10. A disposable filter patch apparatus, comprising:
a filter medium configured to block the passage of a pathogen;
an adhesive seal disposed around an outer border of the filter medium; and
an adhesive protector layer;
wherein the adhesive protector layer is configured to removably cover all of the adhesive seal so as to prevent the adhesive seal from adhering to a surface until the adhesive protector layer is removed;
wherein the filter medium is configured to cover a positive airway pressure mask opening.

11. The apparatus of claim 10, wherein the filter medium is rectangular in shape.

12. The apparatus of claim 11, wherein the adhesive protector layer includes a score spanning a portion of the adhesive protector layer.

13. The apparatus of claim 12, wherein the filter medium comprises a particulate air filter capable of removing pathogens and other contaminants from a user's expelled breath.

14. A method of operating a positive airway pressure device, comprising:
providing a face mask comprising:
a body shaped to fit on a user's face;
an inlet port configured to attach to a gas supply tube;
a mouth portion including a mouth exhaust port;
a nose portion including a nose exhaust port;
a plurality of adjustable straps; and
a seal configured to prevent the escape of air between the body and the user's face;
attaching the inlet port of the face mask to a first end of the gas supply tube, wherein a second end of the gas supply tube is connected to a continuous positive airway pressure device;
disposing a mouth exhaust filter over the mouth exhaust port, wherein the mouth exhaust filter comprises:
a first filter medium;
a first adhesive seal disposed around an outer border of the filter medium; and
a first scored adhesive protector layer removably covering all of the first adhesive seal;
securing, via the adjustable straps, the face mask to the user's face such that the seal prevents the escape of air between the user's face and the body of the face mask; and
activating the continuous positive airway pressure device such that gas actively flows into the face mask via the inlet port.

15. The method of claim 14, wherein the mouth exhaust port includes a first check valve.

16. The method of claim 15, further comprising:
disposing a nose exhaust filter over the nose exhaust port, wherein the nose exhaust filter comprises:
a second filter medium;
a second adhesive seal disposed around an outer border of the second filter medium; and
a second scored adhesive protector layer removably covering all of the second adhesive seal.

17. The method of claim 16, wherein the first and second filter mediums each further comprise a particulate air filter capable of removing pathogens and other contaminants from the user's expelled breath.

18. The method of claim 17, wherein the nose exhaust port includes a second check valve.

19. The method of claim 18, further comprising:
disposing an intake filter over the inlet port, wherein the intake filter comprises:
a third filter medium;
a third adhesive seal disposed around an outer border of the third filter medium; and
a third scored adhesive protector layer removably covering all of the third adhesive seal.

20. The method of claim 19, wherein the third filter medium comprises a particulate air filter capable of removing pathogens and other contaminants from the user's expelled breath.
